(19) United States
(12) Patent Application Publication
(10) Pub. No.: US 2026/0218164 A1
(43) Pub. Date: Jul. 30, 2026

(54) SEPARATION OF LONG OLIGONUCLEOTIDES

(71) Applicant: THERMO FINNIGAN LLC, San Jose, CA (US)

(72) Inventors: Robert L. Ross, Hopkinton, MA (US); Min Du, Acton, MA (US)

(21) Appl. No.: 19/142,671

(22) PCT Filed: Jan. 29, 2024
(86) PCT No.: PCT/US2024/013402

§ 371 Date: Jun. 24, 2025

Related U.S. Application Data
(60) Provisional application No. 63/482,151, filed on Jan. 30, 2023.

(51) Int. Cl. C12N 15/10; G01N 30/72

(52) U.S. Cl. CPC C12N 15/101; G01N 30/7233

(57) ABSTRACT

A method for separation of analyzing oligonucleotides or nucleic acid biopolymers includes loading a sample containing a plurality of oligonucleotide species onto a column; flowing a mobile phase consisting of a combination of a first solution and a second solution through the column to elute the plurality of oligonucleotide species; and analyzing at least one of the first oligonucleotide species and the second oligonucleotide species using a mass spectrometer. The plurality of oligonucleotide species includes a first oligonucleotide species and a second oligonucleotide species. The first solution includes an ion pairing reagent. The ion pairing reagent includes a primary amine or a secondary amine. The proportion of the first solution and the second solution in the mobile phase is varied over time to separate the first oligonucleotide species from the second oligonucleotide species.

### CROSS-REFERENCE TO RELATED APPLICATION

This application is the U.S. National Stage, under 35 U.S. C § 371, of international application PCT/US 2024/013402, having an international filing date of Jan. 29, 2024, which claims priority to U.S. Provisional Application No. 63/482,151, filed on Jan. 30, 2023, the entire contents of which is incorporated herein by reference.

### FIELD

The present disclosure generally relates to the field of chromatography and mass spectrometry including a method for separation of long oligonucleotides.

### INTRODUCTION

Due to recent events, particularly the COVID pandemic, mRNA has been widely adopted as a useful therapy, such as various mRNA based COVID vaccines. Production of non-natural transcripts by in vitro transcription necessitates analytical examination to determine reaction outcome and yield. Ion-pair reversed phase chromatography is widely used for the characterization of proteins and nucleic acids, such as for use as pharmaceuticals. Ion-pair reversed phase chromatography can be coupled with UV as well as mass spectrometry for characterization of the main product as well as impurities in the pharmaceutical product. However, nucleic acids are composed of many structurally and chemically related variants. These variants are often not well resolved. From the foregoing it will be appreciated that a need exists for improved separation methods for long oligonucleotides.

### SUMMARY

In a first aspect, a method for separation of analyzing oligonucleotides or nucleic acid biopolymers, can include loading a sample containing a plurality of oligonucleotide species onto a column. The plurality of oligonucleotide species can include a first oligonucleotide species and a second oligonucleotide species. The method can further include flowing a mobile phase consisting of a combination of a first solution and a second solution through the column to elute the plurality of oligonucleotide species. The first solution can include an ion pairing reagent. The ion pairing reagent can include a primary amine or a secondary amine. The proportion of the first solution and the second solution in the mobile phase can be varied over time to separate the first oligonucleotide species from the second oligonucleotide species. The method can additionally include analyzing at least one of the first oligonucleotide species and the second oligonucleotide species using a mass spectrometer.

In various embodiments of the first aspect, the sample can include the plurality of oligonucleotide species dissolved in the first solution, the second solution, or any combination thereof.

In various embodiments of the first aspect, the second solution can include the ion pairing reagent.

In various embodiments of the first aspect, the ion pairing reagent can include at least 4 carbons. In particular embodiments, the primary amine of the ion pairing reagent can include an alkyl chain of at least 4 carbons, for example, amylamine or hexylamine. In particular embodiments, the secondary amine of the ion pairing reagent can include two alkyl chains, at least one of the alkyl chains including at least 3 carbons, for example, N-ethylpropylamine, dipropylamine (DPA), N-ethylbutylamine, N-propylbutylamine, dibutylamine (DBA), dipentalamine, or dihexylamine.

In various embodiments of the first aspect, the first solution can include a fluoroalcohol, such as hexafluoroisopropanol (HFIP).

In various embodiments of the first aspect, the first solution can include formic acid or formate, acetic acid or acetate, or any combination thereof.

In various embodiments of the first aspect, the first solution can include a first organic solvent, such as methanol, ethanol, acetone, acetonitrile, tetrahydrofuran, isopropanol, or any combination thereof.

In various embodiments of the first aspect, the second solution can include a second organic solvent, such as methanol, ethanol, acetone, acetonitrile, tetrahydrofuran, isopropanol, or any combination thereof.

In various embodiments of the first aspect, the column can include a stationary phase including divinyl benzene or derivatives thereof. In particular embodiments, the stationary phase can be porous with an average pore size of between about 50 Å and about 200 Å and the first and second species can have a size of less than 10 kDa. In particular embodiments, the stationary phase can be porous with an average pore size of between about 200 Å and about 500 Å and the first and second species can have a length of not greater than about 300 nt. In particular embodiments, the stationary phase can be porous with an average pore size of between about 500 Å and about 4000 Å and the first and second species can have lengths of greater than 300 nt.

In various embodiments of the first aspect, the first and second species can have lengths of not greater than about 15000 nt.

In various embodiments of the first aspect, the plurality of oligonucleotide species can be ribonucleic acid (RNA) oligonucleotides. In particular embodiments, the ribonucleic acid (RNA) oligonucleotides can contain modifications. In particular embodiments, the ribonucleic acid (RNA) oligonucleotides can be produced in vitro. In particular embodiments, the ribonucleic acid (RNA) oligonucleotides can be produced in vivo. In particular embodiments, the ribonucleic acid (RNA) oligonucleotides can be encapsulated RNA oligonucleotides.

In a second aspect, a method for separation of analyzing encapsulated oligonucleotides or nucleic acid biopolymers can include solubilizing a sample containing a plurality of encapsulated oligonucleotide species. The plurality of encapsulated oligonucleotide species can include a first encapsulated oligonucleotide species containing a first oligonucleotide species and a second encapsulated oligonucleotide species containing a second oligonucleotide species. Solubilizing the sample can include releasing the first and second oligonucleotide species. The method can also include loading the solubilized sample onto a column; and flowing a mobile phase consisting of a combination of a first solution and a second solution through the column to elute the first and second oligonucleotide species. The first solution can include an ion pairing reagent. The ion pairing reagent can include a primary amine or a secondary amine. The proportion of the first solution and the second solution in the mobile phase can be varied over time to separate the first oligonucleotide species from the second oligonucleotide species. The method can further include analyzing at least one of the first oligonucleotide species and the second oligonucleotide species using a mass spectrometer.

In various embodiments of the second aspect, the sample can include the plurality of oligonucleotide species dissolved in the first solution, the second solution, or any combination thereof.

In various embodiments of the second aspect, second solution can includs the ion pairing reagent.

In various embodiments of the second aspect, the ion pairing reagent can include at least 4 carbons. In particular embodiments, the primary amine of the ion pairing reagent can include an alkyl chains of at least 4 carbons, for example, amylamine or hexylamine. In particular embodiments, the secondary amine of the ion pairing reagent can include two alkyl chains, at least one of the alkyl chains including at least 3 carbons, for example, N-ethylpropylamine, dipropylamine (DPA), N-ethylbutylamine, N-propylbutylamine, dibutylamine (DBA), dipentalamine, or dihexylamine.

In various embodiments of the second aspect, the first solution can include a fluoroalcohol, such as hexafluoroisopropanol (HFIP).

In various embodiments of the second aspect, the first solution can include formic acid or formate, acetic acid or acetate, or any combination thereof.

In various embodiments of the second aspect, the first solution can include a first organic solvent, such as methanol, ethanol, acetone, acetonitrile, tetrahydrofuran, isopropanol, or any combination thereof.

In various embodiments of the second aspect, the second solution can include a second organic solvent, such as methanol, ethanol, acetone, acetonitrile, tetrahydrofuran, isopropanol, or any combination thereof.

In various embodiments of the second aspect, the column can include a stationary phase including divinyl benzene or derivatives thereof. In particular embodiments, the stationary phase can be porous with an average pore size of between about 50 Å and about 200 Å and the first and second species can have a size of less than 10 kDa. In particular embodiments, the stationary phase can be porous with an average pore size of between about 200 Å and about 500 Å and the first and second species can have a length of not greater than about 300 nt. In particular embodiments, the stationary phase can be porous with an average pore size of between about 500 Å and about 4000 Å and the first and second species can have lengths of greater than 300 nt.

In various embodiments of the second aspect, the first and second species can have lengths of not greater than about 15000 nt.

In various embodiments of the second aspect, the first and second oligonucleotide species can be ribonucleic acid (RNA) oligonucleotides.

In various embodiments of the second aspect, the first and second oligonucleotide species can be deoxyribonucleic acid (DNA) oligonucleotides.

In various embodiments of the second aspect, the first and second oligonucleotide species can contain modifications.

In various embodiments of the second aspect, the first and second oligonucleotide species can be produced in vitro.

In various embodiments of the second aspect, the first and second oligonucleotide species can be produced in vivo.

In a third aspect, a method for separation of analyzing viral particles can include solubilizing a sample containing a plurality of viral particle species. The plurality of viral particle species can include a first viral particle species containing a first oligonucleotide species and a second viral particle species containing a second oligonucleotide species. Solubilizing the sample can include releasing the first and second oligonucleotide species. The method can also include loading the solubilized sample onto a column; and flowing a mobile phase consisting of a combination of a first solution and a second solution through the column to elute the first and second oligonucleotide species. The first solution can include an ion pairing reagent. The ion pairing reagent can include a primary amine or a secondary amine. The proportion of the first solution and the second solution in the mobile phase can be varied over time to separate the first oligonucleotide species from the second oligonucleotide species. The method can further include analyzing at least one of the first oligonucleotide species and the second oligonucleotide species using a mass spectrometer.

In various embodiments of the third aspect, the sample can include the plurality of oligonucleotide species dissolved in the first solution, the second solution, or any combination thereof.

In various embodiments of the third aspect, second solution can include the ion pairing reagent.

In various embodiments of the third aspect, the ion pairing reagent can include at least 4 carbons.

In particular embodiments, the primary amine of the ion pairing reagent can include an alkyl chains of at least 4 carbons, for example, amylamine or hexylamine.

In particular embodiments, the secondary amine of the ion pairing reagent can include two alkyl chains, at least one of the alkyl chains including at least 3 carbons, for example N-ethylpropylamine, dipropylamine (DPA), N-ethylbutylamine, N-propylbutylamine, dibutylamine (DBA), dipentalamine, or dihexylamine.

In various embodiments of the third aspect, the first solution can include a fluoroalcohol, such as hexafluoroisopropanol (HFIP).

In various embodiments of the third aspect, the first solution can include formic acid or formate, acetic acid or acetate, or any combination thereof.

In various embodiments of the third aspect, the first solution can include a first organic solvent, such as methanol, ethanol, acetone, acetonitrile, tetrahydrofuran, isopropanol, or any combination thereof.

In various embodiments of the third aspect, the second solution can include a second organic solvent, such as methanol, ethanol, acetone, acetonitrile, tetrahydrofuran, isopropanol, or any combination thereof.

In various embodiments of the third aspect, the column can include a stationary phase including divinyl benzene or derivatives thereof. In particular embodiments, the stationary phase can be porous with an average pore size of between about 50 Å and about 200 Å and the first and second species can have a size of less than 10 kDa. In particular embodiments, the stationary phase can be porous with an average pore size of between about 200 Å and about 500 Å and the first and second species can have a length of not greater than about 300 nt. In particular embodiments, the stationary phase can be porous with an average pore size of between about 500 Å and about 4000 Å and the first and second species can have lengths of greater than 300 nt.

In various embodiments of the third aspect, the first and second species can have lengths of not greater than about 15000 nt.

In various embodiments of the third aspect, the first and second oligonucleotide species can be ribonucleic acid (RNA) oligonucleotides.

In various embodiments of the third aspect, the first and second oligonucleotide species can be deoxyribonucleic acid (DNA) oligonucleotides.

In various embodiments of the third aspect, the first and second oligonucleotides can contain modifications.

### DRAWINGS

For a more complete understanding of the principles disclosed herein, and the advantages thereof, reference is now made to the following descriptions taken in conjunction with the accompanying drawings, in which:

FIGS. 1A, 1B, 1C, and 1D are diagrams illustrating the chemical structure and the positions of potential hydrogen bond donors and hydrogen bond acceptors of RNA nucleosides A, G, C, and U respectively.

FIGS. 2A and 2B are diagrams illustrating the chemical structure and the positions of potential hydrogen bond donors and hydrogen bond acceptors of exemplary tertiary amines triethylamine and diisopropylethylamine respectively.

FIGS. 3A, 3B, and 3C are diagrams illustrating the chemical structure and the positions of potential hydrogen bond donors and hydrogen bond acceptors of exemplary secondary amines diethylamine, dipropylamine, and dibutylamine respectively.

FIGS. 4A and 4B are diagrams illustrating the chemical structure and the positions of potential hydrogen bond donors and hydrogen bond acceptors of exemplary primary amines amylamine and hexylamine respectively.

FIGS. 5A, 5B, and 5C are a flow diagram of method of separating and analyzing oligonucleotides, in accordance with various embodiments.

FIG. 6 is a block diagram of an exemplary chromatography system, in accordance with various embodiments.

FIG. 7 is a block diagram of an exemplary mass spectrometry system, in accordance with various embodiments.

FIG. 8 illustrate the result of analyzing a viral particle, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DESCRIPTION OF VARIOUS EMBODIMENTS

Embodiments of methods for separation of long oligonucleotides are described herein.

The section headings used herein are for organizational purposes only and are not to be construed as limiting the described subject matter in any way.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless described otherwise, all technical and scientific terms used herein have a meaning as is commonly understood by one of ordinary skill in the art to which the various embodiments described herein belongs.

It will be appreciated that there is an implied “about” prior to the temperatures, concentrations, times, pressures, flow rates, cross-sectional areas, etc. discussed in the present teachings, such that slight and insubstantial deviations are within the scope of the present teachings. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the present teachings.

As used herein, “a” or “an” also may refer to “at least one” or “one or more.” Also, the use of “or” is inclusive, such that the phrase “A or B” is true when “A” is true, “B” is true, or both “A” and “B” are true. Further, unless otherwise required by context, singular terms shall include pluralities and plural terms shall include the singular.

A “system” sets forth a set of components, real or abstract, comprising a whole where each component interacts with or is related to at least one other component within the whole.

Ion pair chromatography (IPC) is an effective reversed-phase liquid chromatographic (RPLC) technique for separation of organic ions and partly ionized organic analytes. The technique utilizes the same types of stationary phases and mobile phases as RPLC; the main characteristic for IPC is that one or more ion pair reagents is added to the mobile phase.

The purpose of adding an ion pair reagent to the mobile phase is usually to change the retention time of ionic analytes. By varying the mobile phase concentration of the ion pair reagent, the retention factor for an oppositely charged analyte can be continuously increased by a factor of 10-20 compared to the value with no added ion pair reagent. Correspondingly, it is possible to continuously reduce the retention factor for a similarly charged analyte by a factor of 10-20. The retention factor for non-charged analytes is usually more or less unaffected by the presence of the ion pair reagent.

IPC has been applied in almost all areas of analytical chemistry where chromatography is used. In IPC, water-rich mobile phases can be employed with a variety of buffers and ionic and non-ionic additives and the technique is therefore suitable for separation of important classes of biomolecules and specifically amino acids, peptides, proteins and nucleic acids.

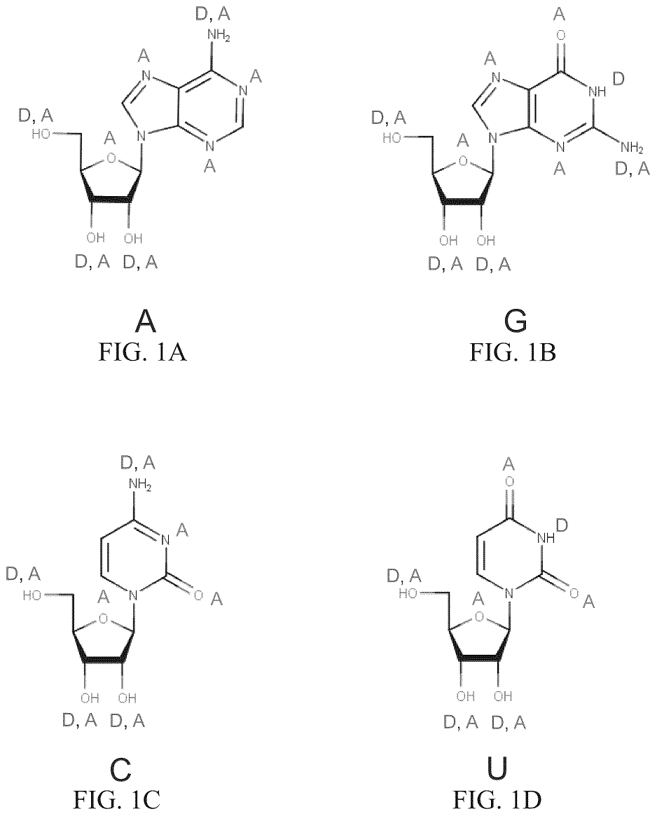

RNA, or ribonucleic acid, is a nucleic acid made up of long chains of nucleotides, which are composed of a sugar, a phosphate group, and a base. There are four different types of bases in RNA: adenine, guanine, cytosine, and uracil. FIGS. 1A-1D illustrate the chemical structures of the nucleosides (ribose sugar and base, excluding the phosphate group) for adenine (FIG. 1A), guanine (FIG. 1B), cytosine (FIG. 1C), and uracil (FIG. 1D). Groups that are available to act as hydrogen bond donors are labeled with a D and groups that are available to act as hydrogen bond acceptors are labeled with an A. Some groups are available to act as either a donor or an acceptor and are labeled with both D and A. As can be seen by the figures, there are a large number of groups that can act as a hydrogen bond acceptor, with a smaller number of groups that can act as a hydrogen bond donor.

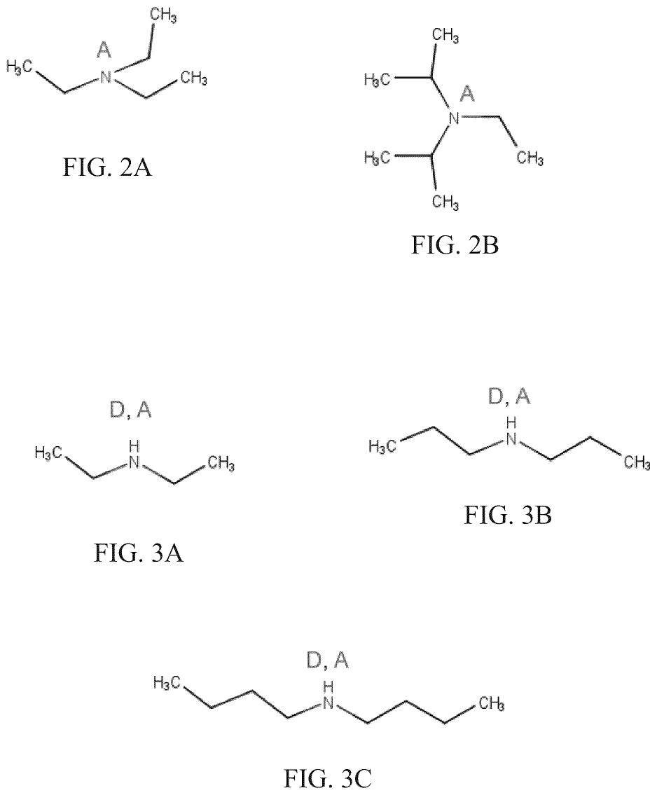

FIGS. 2A and 2B illustrate the structure and positions of potential hydrogen bond donors and hydrogen bond acceptors of exemplary tertiary amines triethylamine (TEA) and diisopropylethylamine (DIPEA) respectively. TEA and DIPEA are commonly used ion pairing reagents. However, as can be seen in the FIGS. 2A and 2B, the tertiary amine can only act as a hydrogen bond acceptor.

FIGS. 3A, 3B, and 3C illustrate the chemical structure and the positions of potential hydrogen bond donors and hydrogen bond acceptors of exemplary secondary amines diethylamine (DEA), dipropylamine (DPA), and dibutylamine (DBA) respectively. As can be seen in the FIGS. 3A, 3B, and 3C, the secondary amine can act as either a hydrogen bond donor or a hydrogen bond acceptor.

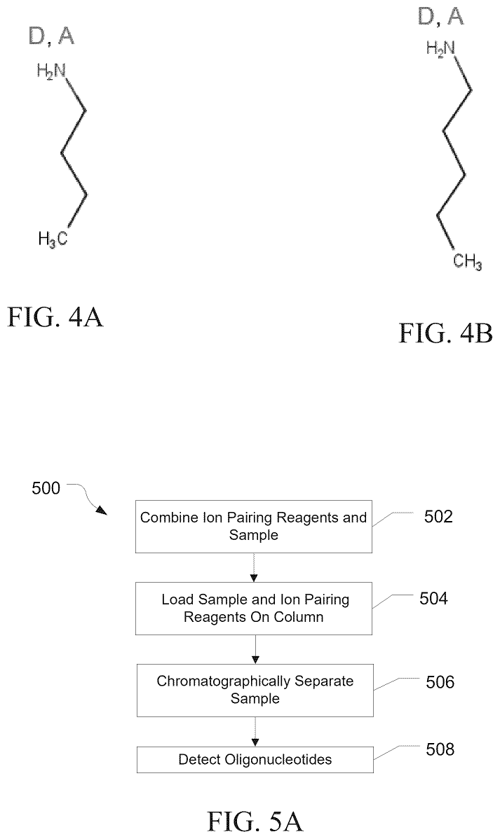

FIGS. 4A and 4B illustrate the chemical structure and the positions of potential hydrogen bond donors and hydrogen bond acceptors of exemplary primary amines amylamine and hexylamine respectively. As can be seen in the FIGS. 4A and 4B, the primary amine can act as either a hydrogen bond donor or a hydrogen bond acceptor.

Using ion pairing reagents that function as a hydrogen bond donors allows for increased interactions with the bases of the nucleic acid, particularly at the Watson-Crick face. Through hydrogen bond type interactions with the bases, retention can be increased. As the number of hydrogen bond acceptor sites increases with length of the nucleic acid, retention can be length dependent, allowing for improved separation of large oligonucleotides. Additionally, because of the differing numbers and arrangement of hydrogen bond acceptor groups for the different bases, there can be differential retention based on the sequence of the oligonucleotides.

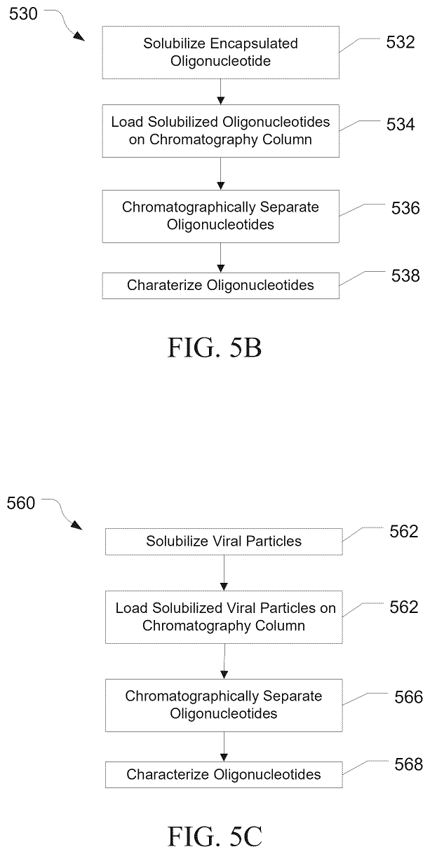

FIG. 5A illustrates an exemplary method 500 of analyzing a sample containing a plurality of oligonucleotide species. In various embodiments, the oligonucleotide species can include ribonucleic acid (RNA) oligonucleotides or deoxyribonucleic acid (DNA) oligonucleotides. The oligonucleotides can contain natural or non-natural modifications. The oligonucleotides can be produced in vitro or in vivo. In various embodiments, the oligonucleotides can be encapsulated RNA oligonucleotides that are encapsulated in structures containing lipids, proteins, or any combination thereof.

At 502, the ion pairing reagent can be combined with a sample. In various embodiments, the sample can be dissolved or resuspended in a first solution, a second solution, or any combination thereof. The ion pairing reagent can include a primary amine or a secondary amine. In various embodiments, the ion pairing reagent can include at least about 4 carbons, but generally not more than about 16 carbons.

A primary amine ion pairing reagent can include an alkyl chain of at least 4 carbons. In particular embodiments, the primary amine ion pairing reagent can include amylamine or hexylamine.

A secondary amine ion pairing reagent can include two alkyl chains, at least one of which contains at least 3 carbons, but generally less than 8 carbons, such as N-ethylpropylamine, dipropylamine (DPA), N-ethylbutylamine, N-propylbutylamine, dibutylamine (DBA), dipentalamine, or dihexylamine.

At 504, the sample and the ion pairing reagents can be loaded onto a chromatography column. In various embodiments, the chromatography column can include a stationary phase including divinyl benzene or derivatives thereof. In various embodiments, the station phase can be porous, such as having an average pore size of at least about 50 Å, such as at least about 200 Å, even at least about 500 Å. The choice of average pore size can be affected by the size of oligonucleotides to be separated. A stationary phase with an average pore size of between about 50 Å and about 200 Å can be used to separate oligonucleotides having a size of less than 10 kDa. A stationary phase having an average pore size of between about 200 Å and about 500 Å can be used to separate oligonucleotides of not greater than about 300 nt. A stationary phase having an average pore size of between about 500 Å and about 4000 Å and can be used to separate oligonucleotides of greater that about 300 nt, but generally not greater than about 15000 nt.

At 506, an eluent can be applied to the column to elute the plurality of oligonucleotide species. Generally, it is advantageous for the solution conditions of the sample when applied to the column to be close to the starting conditions for the eluent gradient. In various embodiments, the eluent can include a combination of the first solution and a second solution. In particular embodiments, the ratio of the first solution to the second solution can be varies to form a gradient of solvent conditions to cause the separation of the various oligonucleotide species, such as a first oligonucleotide species from a second oligonucleotide species, based upon differential retention within the column.

In various embodiments, only the first solution and second solution can vary in the concentration of the ion pairing reagent, such that the gradient of solvent conditions includes a gradient of ion pairing reagent concentration. For example, the second solution can include more or less of the ion pairing reagent than the first solution, even only the first solution including the ion pairing reagent and the second solution including none. In other embodiments, the first and second solution can include the same concentration of ion pairing reagent such that the concentration of ion pairing reagent is substantially constant through the separation. In various embodiments the concentration of the ion pairing reagent can be between about 1 mM and about 500 mM, such as between about 5 mM and about 500 mM.

In various embodiments, the first solution can include a fluoroalcohol, such as hexafluoroisopropanol (HFIP). In various embodiments the fluoroalcohol can be in a concentration of between about 5 mM and about 500 mM. In alternate embodiments, a fluoroalcohol may not be included. In various embodiments the first solution can include formic acid or formate, acetic acid or acetate, or any combination thereof, such as to achieve a pH in a range between about 2 and about 11, such as about 4 to 10. In alternate embodiments, the first solution may not include formic acid or formate, acetic acid or acetate. In various embodiments, the first solution can include a first organic solvent, such as methanol, ethanol, acetone, acetonitrile, tetrahydrofuran, isopropanol, or any combination thereof. In various embodiments, the organic solvent can be in a concentration of between about 0% and about 100%. When less than about 100%, the remaining amount of the solution can include water.

In various embodiments, the second solution can include similar or identical components to the first solution but in different concentrations. For example, the second solution can include a fluoroalcohol, formic acid, formate, acetic acid, acetate, and/or an organic solvent. In other embodiments, the second solution may contain at least some components different from the first solution.

At 508, the oligonucleotide species can be detected using a detector, such as a UV/VIS detector or a mass spectrometer. Additionally, the sample can be analyzed based on the signal observed by the detector and the retention time to identify an oligonucleotide species and/or quantify an amount of an oligonucleotide species.

FIG. 5B illustrates an exemplary method 530 of analyzing a sample containing a plurality of encapsulated oligonucleotide species. In various embodiments, the encapsulated RNA oligonucleotides can be encapsulated in structures containing lipids, proteins, or any combination thereof. In various embodiments, the encapsulated oligonucleotide species can include ribonucleic acid (RNA) oligonucleotides or deoxyribonucleic acid (DNA) oligonucleotides. The oligonucleotides can contain natural or non-natural modifications. The oligonucleotides can be produced in vitro or in vivo.

At 532, the ion pairing reagent can be combined with a sample. In various embodiments, the sample can be dissolved or resuspended in a first solution, a second solution, or any combination thereof. In various embodiments, dissolving or resuspending the sample can dissolve the lipids and/or denature the proteins of the encapsulating structure such that the oligonucleotides contained therein are released into the solution.

The ion pairing reagent can include a primary amine or a secondary amine. In various embodiments, the ion pairing reagent can include at least about 4 carbons, but generally not more than about 16 carbons.

A primary amine ion pairing reagent can include an alkyl chain of at least 4 carbons. In particular embodiments, the primary amine ion pairing reagent can include amylamine or hexylamine.

A secondary amine ion pairing reagent can include two alkyl chains, at least one of which contains at least 3 carbons, but generally less than 8 carbons, such as N-ethylpropylamine, dipropylamine (DPA), N-ethylbutylamine, N-propylbutylamine, dibutylamine (DBA), dipentalamine, or dihexylamine.

At 534, the sample and the ion pairing reagents can be loaded onto a chromatography column. In various embodiments, the chromatography column can include a stationary phase including divinyl benzene or derivatives thereof. In various embodiments, the station phase can be porous, such as having an average pore size of at least about 50 Å, such as at least about 200 Å, even at least about 500 Å. The choice of average pore size can be affected by the size of oligonucleotides to be separated. A stationary phase with an average pore size of between about 50 Å and about 200 Å can be used to separate oligonucleotides having a size of less than 10 kDa. A stationary phase having an average pore size of between about 200 Å and about 500 Å can be used to separate oligonucleotides of not greater than about 300 nt. A stationary phase having an average pore size of between about 500 Å and about 4000 Å and can be used to separate oligonucleotides of greater that about 300 nt, but generally not greater than about 15000 nt.

At 536, an eluent can be applied to the column to elute the plurality of oligonucleotide species. Generally, it is advantageous for the solution conditions of the sample when applied to the column to be close to the starting conditions for the eluent gradient. In various embodiments, the eluent can include a combination of the first solution and a second solution. In particular embodiments, the ratio of the first solution to the second solution can be varies to form a gradient of solvent conditions to cause the separation of the various oligonucleotide species, such as a first oligonucleotide species from a second oligonucleotide species, based upon differential retention within the column.

In various embodiments, the first solution and second solution can differ in the concentration of the ion pairing reagent, such that the gradient of solvent conditions includes a gradient of ion pairing reagent concentration. For example, the second solution can include more or less of the ion pairing reagent than the first solution, even only the first solution including the ion pairing reagent and the second solution including none. In other embodiments, the first and second solution can include the same concentration of ion pairing reagent such that the concentration of ion pairing reagent is substantially constant through the separation. In various embodiments the concentration of the ion pairing reagent can be between about 5 mM and about 500 mM.

In various embodiments, the first solution can include a fluoroalcohol, such as hexafluoroisopropanol (HFIP). In various embodiments the fluoroalcohol can be in a concentration of between about 5 mM and about 500 mM. In various embodiments the first solution can include formic acid or formate, acetic acid or acetate, or any combination thereof, such as to achieve a pH in a range between about 2 and about 11, such as about 4 to 10. In various embodiments, the first solution can include a first organic solvent, such as methanol, ethanol, acetone, acetonitrile, tetrahydrofuran, isopropanol, or any combination thereof. In various embodiments, the organic solvent can be in a concentration of between about 0% and about 100%. When less than about 100%, the remaining amount of the solution can include water.

In various embodiments, the second solution can include similar or identical components to the first solution but in different concentrations. For example, the second solution can include a fluoroalcohol, formic acid, formate, acetic acid, acetate, and/or an organic solvent. In other embodiments, the second solution may contain at least some components different from the first solution.

At 538, the oligonucleotide species can be detected using a detector, such as a UV/VIS detector or a mass spectrometer. Additionally, the sample can be analyzed based on the signal observed by the detector and the retention time to identify an oligonucleotide species and/or quantify an amount of an oligonucleotide species.

FIG. 5C illustrates an exemplary method 560 of analyzing a sample containing a plurality of viral particles. The viral particles can include lipid and/or protein structures enclosing oligonucleotides. In various embodiments, the oligonucleotide species can include ribonucleic acid (RNA) oligonucleotides or deoxyribonucleic acid (DNA) oligonucleotides. The oligonucleotides can contain natural or non-natural modifications. The oligonucleotides can be produced in vitro or in vivo.

At 562, the ion pairing reagent can be combined with a sample. In various embodiments, the sample can be dissolved or resuspended in a first solution, a second solution, or any combination thereof. In various embodiments, dissolving or resuspending the sample can solubilize the viral particles by dissolving the lipids and/or denaturing the proteins of the viral particles such that oligonucleotides contained within the viral particles are released into the solution.

The ion pairing reagent can include a primary amine or a secondary amine. In various embodiments, the ion pairing reagent can include at least about 4 carbons, but generally not more than about 16 carbons.

A primary amine ion pairing reagent can include an alkyl chain of at least 4 carbons. In particular embodiments, the primary amine ion pairing reagent can include amylamine or hexylamine.

A secondary amine ion pairing reagent can include two alkyl chains, at least one of which contains at least 3 carbons, but generally less than 8 carbons, such as N-ethylpropylamine, dipropylamine (DPA), N-ethylbutylamine, N-propylbutylamine, dibutylamine (DBA), dipentalamine, or dihexylamine.

At 564, the sample and the ion pairing reagents can be loaded onto a chromatography column. In various embodiments, the chromatography column can include a stationary phase including divinyl benzene or derivatives thereof. In various embodiments, the station phase can be porous, such as having an average pore size of at least about 50 Å, such as at least about 200 Å, even at least about 500 Å. The choice of average pore size can be affected by the size of oligonucleotides to be separated. A stationary phase with an average pore size of between about 50 Å and about 200 Å can be used to separate oligonucleotides having a size of less than 10 kDa. A stationary phase having an average pore size of between about 200 Å and about 500 Å can be used to separate oligonucleotides of not greater than about 300 nt. A stationary phase having an average pore size of between about 500 Å and about 4000 Å and can be used to separate oligonucleotides of greater that about 300 nt, but generally not greater than about 15000 nt.

At 566, an eluent can be applied to the column to elute the plurality of oligonucleotide species. Generally, it is advantageous for the solution conditions of the sample when applied to the column to be close to the starting conditions for the eluent gradient. In various embodiments, the eluent can include a combination of the first solution and a second solution. In particular embodiments, the ratio of the first solution to the second solution can be varies to form a gradient of solvent conditions to cause the separation of the various oligonucleotide species, such as a first oligonucleotide species from a second oligonucleotide species, based upon differential retention within the column.

In various embodiments, only the first solution and second solution can vary in the concentration of the ion pairing reagent, such that the gradient of solvent conditions includes a gradient of ion pairing reagent concentration. For example, the second solution can include more or less of the ion pairing reagent than the first solution, even only the first solution including the ion pairing reagent and the second solution including none. In other embodiments, the first and second solution can include the same concentration of ion pairing reagent such that the concentration of ion pairing reagent is substantially constant through the separation. In various embodiments the concertation of the ion pairing reagent can be between about 5 mM and about 500 mM.

In various embodiments, the first solution can include a fluoroalcohol, such as hexafluoroisopropanol (HFIP). In various embodiments the fluoroalcohol can be in a concentration of between about 5 mM and about 500 mM. In various embodiments the first solution can include formic acid or formate, acetic acid or acetate, or any combination thereof, such as to achieve a pH in a range between about 2 and about 10. In various embodiments, the first solution can include a first organic solvent, such as methanol, ethanol, acetone, acetonitrile, tetrahydrofuran, isopropanol, or any combination thereof. In various embodiments, the organic solvent can be in a concentration of between about 0% and about 100%. When less than about 100%, the remaining amount of the solution can include water.

In various embodiments, the second solution can include similar or identical components to the first solution but in different concentrations. For example, the second solution can include a fluoroalcohol, formic acid, formate, acetic acid, acetate, and/or an organic solvent. In other embodiments, the second solution may contain at least some components different from the first solution.

At 568, the oligonucleotide species can be detected using a detector, such as a UV/VIS detector or a mass spectrometer. Additionally, the sample can be analyzed based on the signal observed by the detector and the retention time to identify an oligonucleotide species and/or quantify an amount of an oligonucleotide species.

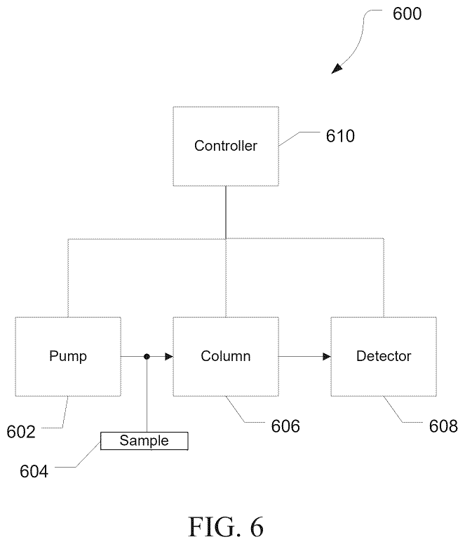

FIG. 6 depicts a liquid chromatography system 600 according to one aspect of the invention. The liquid chromatography system 600 comprises an analytical pump 602 to pump a solvent through the system 600. The system 600 comprises a sample reservoir 604 comprising a sample to be analysed. The system 600 further comprises a separation column 606 and a detector 608. System 600 also comprises a controller 610.

The liquid chromatography system 600 is adapted to retrieve a sample from the sample reservoir 604. The sample can then be introduced into the system

The liquid chromatography system 1000 is further adapted to introduce the sample into the separation column 606.

The system 600 is also adapted to inject the sample into the separation column 606 by means of the analytical flow. This can be done by guiding the sample by means of the analytical pump 602. The separation column 606 can separate the sample into component species based on retention time within the separation column 606. After separation of the sample by the separation column 606, the separated components can be detected by a detector 608. In some embodiments, the detector 608 can be an optical detector, such as an absorption detector, refractive index detector, a fluorescence detector, or the like. In other embodiments, the detector 608 can be a conductivity detector or an electrochemical detector. In yet other embodiments, the detector 608 can be a mass spectrometer.

In various embodiments, the separation column 606 generally consists of a tube packed with a stationary phase medium. The stationary phase medium can affect the time it takes for a compound to travel through the column (retention time). The effect can be different for different compounds, such that individual components of a sample can be separated based on their respective retention times. There are a variety of stationary phase mediums, including porous materials, ionic materials, polar materials, non-polar materials, and the like. Porous materials can affect retention time based on the size of a molecule and the ability of the molecule to enter the porous material. Ionic materials can affect retention time based on charge attraction or repulsion between the ionic material and the compounds. Polar and non-polar materials can affect retention time based on the hydrophobicity or hydrophilicity of the compounds.

In various embodiments, nucleotides and nucleosides can be separated using a reverse phase separation in which a hydrophobic non-polar stationary phase material is used along with mobile phase with varying hydrophobicity depending on the ratio of polar to organic solvents. For example, a C18 column can be used with an ammonium acetate or ammonium formate buffer system to separate the nucleosides. In particular embodiments, an aqueous mobile phase of 5 mM ammonium acetate at a pH of about 5 can be used and a gradient of increasing concentrations of acetonitrile (up to about 40%) or methanol (up to about 50%) can be used to separate the nucleosides. Suitable columns and buffer systems would be apparent to one of skill in the art and are within the scope of this disclosure.

In other embodiments, nucleotides and nucleosides can be separated using hydrophilic interaction liquid chromatography (HILIC) in which a hydrophilic stationary phase material is used along with hydrophobic mobile phase, such as acetonitrile. Suitable columns and buffer systems would be apparent to one of skill in the art and are within the scope of this disclosure.

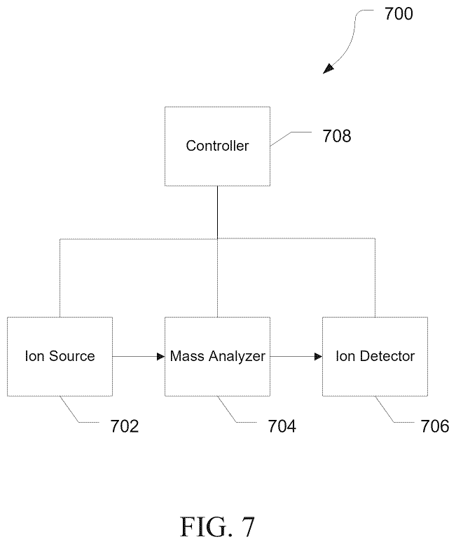

Various embodiments of mass spectrometry platform 700 can include components as displayed in the block diagram of FIG. 7. In various embodiments, mass spectrometry platform 700 can operate as a detector 608 of system 600. In various embodiments, elements of FIG. 7 can be incorporated into mass spectrometry platform 700. According to various embodiments, mass spectrometer 700 can include an ion source 702, a mass analyzer 704, an ion detector 706, and a controller 708.

In various embodiments, the ion source 702 generates a plurality of ions from a sample. The ion source can include, but is not limited to, a matrix assisted laser desorption/ionization (MALDI) source, electrospray ionization (ESI) source, atmospheric pressure chemical ionization (APCI) source, atmospheric pressure photoionization source (APPI), inductively coupled plasma (ICP) source, electron ionization source, chemical ionization source, photoionization source, glow discharge ionization source, thermospray ionization source, and the like.

In various embodiments, the mass analyzer 704 can separate ions based on a mass-to-charge ratio of the ions. For example, the mass analyzer 704 can include a quadrupole mass filter analyzer, a quadrupole ion trap analyzer, a time-of-flight (TOF) analyzer, an electrostatic trap (e.g., Orbitrap) mass analyzer, Fourier transform ion cyclotron resonance (FT-ICR) mass analyzer, and the like. In various embodiments, the mass analyzer 704 can also be configured to fragment the ions using collision induced dissociation (CID) electron transfer dissociation (ETD), electron capture dissociation (ECD), photo induced dissociation (PID), surface induced dissociation (SID), and the like, and further separate the fragmented ions based on the mass-to-charge ratio.

In various embodiments, the ion detector 706 can detect ions. For example, the ion detector 706 can include an electron multiplier, a Faraday cup, and the like. Ions leaving the mass analyzer can be detected by the ion detector. In various embodiments, the ion detector can be quantitative, such that an accurate count of the ions can be determined. In various embodiments, such as for an electrostatic trap (e.g. ORBITRAP) mass analyzer, the mass analyzer 704 and the ion detector 706 can be combined into a single device.

In various embodiments, the controller 708 can communicate with the ion source 702, the mass analyzer 704, and the ion detector 706. For example, the controller 708 can configure the ion source or enable/disable the ion source. Additionally, the controller 708 can configure the mass analyzer 704 to select a particular mass range to detect. Further, the controller 708 can adjust the sensitivity of the ion detector 706, such as by adjusting the gain. Additionally, the controller 708 can adjust the polarity of the ion detector 706 based on the polarity of the ions being detected. For example, the ion detector 706 can be configured to detect positive ions or be configured to detected negative ions.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

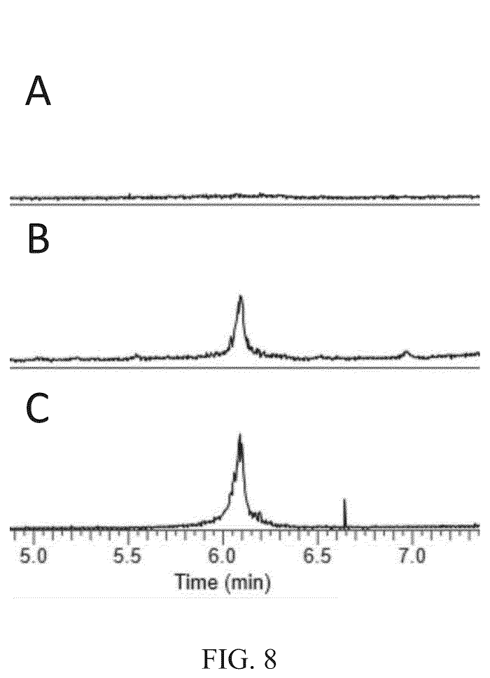

Example 1 is an analysis nucleic acid encapsulated within a bacteriophage. Figure A is a blank injection. FIG. 8B shows the results of injecting the encapsulated RNA within the bacteriophage. FIG. 8C shows the results of an injection of the purified RNA extracted from the bacteriophage.

1. A method for separation of analyzing oligonucleotides or nucleic acid biopolymers, comprising:
loading a sample containing a plurality of oligonucleotide species onto a column, the column including a stationary phase including divinyl benzene or derivatives thereof, the plurality of oligonucleotide species including a first oligonucleotide species and a second oligonucleotide species;
flowing a mobile phase consisting of a combination of a first solution and a second solution through the column to elute the plurality of oligonucleotide species, the first solution including an ion pairing reagent, the ion pairing reagent including a primary amine or a secondary amine, wherein the proportion of the first solution and the second solution in the mobile phase is varied over time to separate the first oligonucleotide species from the second oligonucleotide species; and
analyzing at least one of the first oligonucleotide species and the second oligonucleotide species using a mass spectrometer.

2-16. (canceled)

17. The method of claim 1 wherein the stationary phase is porous with an average pore size of between about 50 Å and about 200 Å and the first and second species having a size of less than 10 kDa.

18. The method of claim 1 wherein the stationary phase is porous with an average pore size of between about 200 Å and about 500 Å and the first and second species having a length of not greater than about 300 nt.

19. The method of claim 1 wherein the stationary phase being porous with an average pore size of between about 500 Å and about 4000 Å and the first and second species having lengths of greater than 300 nt.

20. The method of claim 1 wherein the first and second species have lengths of not greater than about 15000 nt.

21. The method of claim 1 wherein the plurality of oligonucleotide species are ribonucleic acid (RNA) oligonucleotides.

22. The method of claim 21 wherein the ribonucleic acid (RNA) oligonucleotides contain modifications.

23. The method of claim 21 wherein the ribonucleic acid (RNA) oligonucleotides are produced in vitro or in vivo.

24. (canceled)

25. The method of claim 21 wherein the ribonucleic acid (RNA) oligonucleotides are encapsulated RNA oligonucleotides.

26. A method for separation of analyzing encapsulated oligonucleotides or nucleic acid biopolymers, comprising:
solubilizing a sample containing a plurality of encapsulated oligonucleotide species, the plurality of encapsulated oligonucleotide species including a first encapsulated oligonucleotide species containing a first oligonucleotide species and a second encapsulated oligonucleotide species containing a second oligonucleotide species, wherein solubilizing the sample includes releasing the first and second oligonucleotide species;
loading the solubilized sample onto a column, the column includes a stationary phase including divinyl benzene or derivatives thereof;
flowing a mobile phase consisting of a combination of a first solution and a second solution through the column to elute the first and second oligonucleotide species, the first solution including an ion pairing reagent, the ion pairing reagent including a primary amine or a secondary amine, wherein the proportion of the first solution and the second solution in the mobile phase is varied over time to separate the first oligonucleotide species from the second oligonucleotide species; and
analyzing at least one of the first oligonucleotide species and the second oligonucleotide species using a mass spectrometer.

27-41. (canceled)

42. The method of claim 26 wherein the stationary phase is porous with an average pore size of between about 50 Å and about 200 Å and the first and second species having a size of less than 10 kDa.

43. The method of claim 26 wherein the stationary phase is porous with an average pore size of between about 200 Å and about 500 Å and the first and second species having a length of not greater than about 300 nt.

44. The method of claim 26 wherein the stationary phase being porous with an average pore size of between about 500 Å and about 4000 Å and the first and second species having lengths of greater than 300 nt.

45. The method of claim 26 wherein the first and second species have lengths of not greater than about 15000 nt.

46. The method of claim 26 wherein the first and second oligonucleotide species are ribonucleic acid (RNA) oligonucleotides.

47. The method of claim 26 wherein the first and second oligonucleotide species are deoxyribonucleic acid (DNA) oligonucleotides.

48. The method of claim 26 wherein the first and second oligonucleotide species contain modifications.

49. The method of claim 26 wherein the first and second oligonucleotide species are produced in vitro or in vivo.

50. (canceled)

51. A method for separation of analyzing viral particles, comprising:
solubilizing a sample containing a plurality of viral particle species, the plurality of viral particle species including a first viral particle species containing a first oligonucleotide species and a second viral particle species containing a second oligonucleotide species, wherein solubilizing the sample includes releasing the first and second oligonucleotide species;
loading the solubilized sample onto a column, the column includes a stationary phase including divinyl benzene or derivatives thereof;
flowing a mobile phase consisting of a combination of a first solution and a second solution through the column to elute the first and second oligonucleotide species, the first solution including an ion pairing reagent, the ion pairing reagent including a primary amine or a secondary amine, wherein the proportion of the first solution and the second solution in the mobile phase is varied over time to separate the first oligonucleotide species from the second oligonucleotide species; and
analyzing at least one of the first oligonucleotide species and the second oligonucleotide species using a mass spectrometer.

52-66. (Canceled)

67. The method of claim 51 wherein the stationary phase is porous with an average pore size of between about 50 Å and about 200 Å and the first and second species having a size of less than 10 kDa.

68. The method of claim 51 wherein the stationary phase is porous with an average pore size of between about 200 Å and about 500 Å and the first and second species having a length of not greater than about 300 nt.

69. The method of claim 51 wherein the stationary phase being porous with an average pore size of between about 500 Å and about 4000 Å and the first and second species having lengths of greater than 300 nt.

70. The method of claim 51 wherein the first and second species have lengths of not greater than about 15000 nt.

71. The method of claim 51 wherein the first and second oligonucleotide species are ribonucleic acid (RNA) oligonucleotides.

72. The method of claim 51 wherein the first and second oligonucleotide species are deoxyribonucleic acid (DNA) oligonucleotides.

73. The method of claim 51 wherein the first and second oligonucleotides contain modifications.
